(19) United States
(12) Patent Application Publication
(10) Pub. No.: US 2009/0029401 A1
(43) Pub. Date: Jan. 29, 2009

(54) Method for detecting biofuel producing microbes

(75) Inventors: Ian Fotheringham, Edinburgh (GB); Nicole Kaftzik, Roslin (DE); Nicholas Oswald, Edinburgh (GB)

Correspondence Address: ROCKEY, DEPKE & LYONS, LLC, SEARS TOWER, SUITE 5450, CHICAGO, IL, 60606-6306, US

(73) Assignee: Richmond Chemical Corporation

(21) Appl. No.: 12/220,344

(22) Filed: Jul. 24, 2008

Related U.S. Application Data
(60) Provisional application No. 60/961,896, filed on Jul. 25, 2007.

(51) Int. Cl. C12Q 1/28

(52) U.S. Cl. 435/28

(57) ABSTRACT

A method for detecting biofuel producing microbes in a colorimetric assay by providing a microbe, a feedstock, and an assay solution. The assay solution contains an alcohol oxidase, a peroxidase, and a peroxidase co-substrate. The microbe is cultured with the feedstock as part of a culture medium that produces a detectable amount of bioalcohol. The culture medium is contacted with the assay solution to produce a colorimetric reaction to screen for a biofuel producing microbe.

### CROSS-REFERENCE TO RELATED APPLICATIONS

This application claims priority to U.S. Provisional Patent Application Ser. No. 60/961,896 filed Jul. 25, 2007, the entire disclosure of which is incorporated herein by reference. Priority to this application is claimed under 35 U.S.C. §§119 and/or 120.

### FIELD OF THE INVENTION

The present invention is generally related to a method for detecting biofuel producing microbes. More particularly, the present invention is directed to a method of screening biofuel producing microbes using a colorimetric assay.

### BACKGROUND

The need for alternative energy sources is of increasing importance in today's society. Concerns over rising oil prices, environmental hazards and global warming are driving industries to seek alternatives to non-renewable energy sources such as fossil fuels, and turning to renewable energy sources, namely biofuels. Ethanol, propanol, and butanol are alcohols commonly produced in microorganisms through the fermentation process. These biologically produced alcohols, also referred to as bioalcohols, offer a valuable source for biofuels, particularly as fuel components for gasoline. The United States (U.S.) made a major commitment to the development of bioethanol, with its announcement in January of 2006 that the U.S. would focus on producing bioethanol and other biofuel resources to reduce America's dependence on fossil fuels. The U.S. has become the world's largest producer of bioethanol, and the number of bioethanol plants and E85 filling stations continues to grow significantly.

At present in the U.S. bioethanol is produced by the microbial fermentation of glucose released from biomass. Corn is the primary biomass used in U.S. bioethanol production, however bioethanol can also be produced from other U.S. crops such as wheat, barley and sugar beet. Additionally, second-generation bioethanol production is currently being developed. In the future, lignocellulosic biomass such as organic waste, bagasse, wood and switch-grass may be used to produce bioethanol, but these processes cannot yet be used on a commercial scale. Current problems with the production of bioethanol and other biofuels, such as biobutanol, are attributable to the lack of efficiency with which currently available microbes extract and utilize sugars of biomass sources; therefore affecting the yield of bioalcohols which can be achieved from a given feedstock.

Consequently, there is a need to produce superior microbes by genetic manipulation in order to facilitate more complete, more efficient and more versatile processing of available biomass to produce ethanol and other non-fossil fuels. Historically, the most efficient microbes used for industrial processes result from intensive, high-throughput screening techniques that enable many hundreds of thousands or millions of individual microbial isolates to be rapidly assessed in order to identify isolates which, by natural or induced mutation processes, have acquired superior properties suitable for efficient and versatile industrial application. In the case of biofuel producing organisms, such screens are not readily available. The present invention describes a rapid, versatile and extremely high throughput colorimetric assay by which microbes can be rapidly identified possessing superior, desirable properties to improve current production of bioethanol and other biofuels. The present invention is provided to overcome limitations and drawbacks of the prior art, and to provide novel aspects not heretofore available.

### SUMMARY OF THE INVENTION

The present invention is directed to a method for detecting biofuel producing microbes. One aspect of the present invention is to provide a microbe and an assay solution. The assay solution contains an alcohol oxidase (ALOX), a peroxidase, and a peroxidase co-substrate. The biofuel, such as ethanol, produced by the microbe is contacted with the assay solution to produce a colorimetric reaction thereby allowing direct screening for the presence of a biofuel producing microbe.

A second aspect of the present invention is to provide a method for detecting biofuel producing microbes by providing a microbe, a carbon containing feedstock and an assay solution. The assay solution contains an alcohol oxidase, a peroxidase, and a peroxidase co-substrate. The microbe is cultured in the feedstock as part of a culture medium. The culture medium is contacted with the assay solution to produce a colorimetric reaction to screen for biofuel producing microbes. A third aspect of the present invention is to provide a colorimetric assay for screening for biofuel producing microbes that does not react with the feedstock.

A further aspect of the present invention is to provide a microbe and an assay solution. The assay solution contains an alcohol oxidase, a peroxidase, and a peroxidase co-substrate. The microbe is contacted with the assay solution to produce a detectable amount of hydrogen peroxide. The hydrogen peroxide is detected by reaction with the peroxidase and the peroxidase co-substrate, generating a colorimetric reaction product that enables screening for a biofuel producing microbe.

Another aspect of the present invention is to provide a kit for detecting the presence of biofuel producing microbes in which the kit has a container containing an assay solution having an alcohol oxidase, a peroxidase, and a peroxidase co-substrate. The kit further includes a set of instructions on how to use the kit in accordance with the present invention.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 is a graphical representation of alcohol oxidase (ALOX) assayed against various alcohols (10% v/v) using a colorimetric assay and 4-aminoantipyrine/2,4,6-trobromo-3-hydroxybenzoic acid (4-AAP/TBHBA) as the Horseradish Peroxidase (HRP) co-substrate described by the present invention. Activity is shown in mAbs/min for two different alcohol oxidase concentrations. No activity is detected towards glycerol (glycerine) or glucose (not shown in the graph), which serve as microbial feedstocks for biofuel production.

FIG. 2 is a graphical representation of alcohol oxidase (ALOX) assayed against various alcohols (10% v/v) using a calorimetric screen and 2,2′-azino-bis(3-ethylbenzthiazoline-6-sulfonic acid) ABTS as the HRP co-substrate described by the present invention. Activity is shown in mAbs/min using an alcohol oxidase concentration of 0.1 U/ml. No activity is detected towards glycerol (glycerine) or glucose (not shown in the graph), which serve as microbial feedstocks for biofuel production.

FIG. 3 is a graphical representation of alcohol oxidase (ALOX) assayed against ethanol at different concentrations (0-10% v/v) using a colorimetric screen and 4-AAP/TBHBA as the HRP co-substrate described by the present invention. Activity is shown in mAbs/min for an alcohol oxidase concentration of 1 U/ml.

### DETAILED DESCRIPTION

The present invention is capable of embodiments in many different forms. Preferred embodiments of the invention are disclosed with the understanding that the present disclosure is to be considered as exemplifications of the principles of the invention and are not intended to limit the broad aspects of the invention to the embodiments illustrated.

The present invention discloses a direct enzymatic, color based (colorimetric) screen capable of detecting microbes, particularly bacteria or yeast, which produce bioalcohols, such as ethanol or butanol, by fermentation of a variety of carbon containing feedstocks including, but not limited to, glucose, cellulosic biomass and glycerol (sometimes referred to as glycerin). The screen of the present invention can further be adapted for future applications with enzyme evolution to detect alternate valuable products such as, but not limited to, 1,2 propanediol or 1,3-propanediol, also produced by microbial fermentation of a variety of carbon sources.

The present invention further discloses using the enzyme alcohol oxidase, from yeast, such as Pichia pastoris or Pichia angusta, in a calorimetric screen to directly detect the presence or appearance of bioalcohols, such as but not limited to methanol, ethanol, butanol, 1,2 propanediol, and 1,3-propanediol. The calorimetric screen is quantitative; therefore it can determine the rate and extent of bioalcohol production. The calorimetric screen of the present invention can be used in a liquid phase assay form, such as in a 96 well microtitre plate. More significantly, the present invention can be used in a solid phase format on a solid assay media in a Petri plate on which colonies of microbes such as bacteria or yeast can be grown. In the solid phase assay, the colorimetric screen is capable of detecting “mutant” microbes that are characterized as superior bioalcohol producers. Such mutant microbes may have a faster rate of bioalcohol production or greater overall accumulation (or reduced inhibition of production) of products such as methanol, ethanol, butanol, 1,2-propanediol, or 1,3-propanediol for biofuel and other chemical applications from biomass derived carbon sources.

The method of the present invention for detecting and screening bioalcohol producing microbes relies on the following reaction. Alcohol oxidase catalyzes the oxidation of short-chain, primary, aliphatic alcohols to their respective aldehydes thereby generating hydrogen peroxide (H₂O₂). The alcohol oxidase enzyme has the highest affinity for methanol, followed by ethanol, with its affinity decreasing with the increasing chain length of the alkyl group.

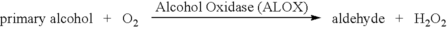

Consequently, alcohol oxidase is a significant enzyme required for detecting biofuel production in microbes as disclosed by the present invention. In one embodiment, alcohol oxidase from Pichia Pastoris (Sigma) Product number: A2404 is preferred. An alcohol oxidase solution can be prepared from Pichia pastoris, aqueous phosphate-buffered 30% sucrose solution, 30 unit/mg protein, 44 mg/ml, 1.32 U/μl. The present invention contemplates using other sources of alcohol oxidases known in the art such as, but not limited to, yeast, and more specifically to Pichia, Pichia pastoris, Pichia angusta and Saccharomyces.

Microbes such as yeast or bacteria can be cultured under conditions described in the art to produce biofuels, such as ethanol, by fermentation of glucose, glycerol or other carbon source containing feedstocks. Production of biofuels by microbes can be carried out under laboratory conditions either in liquid culture or on a solid medium, such as nutrient agar, by culturing suitable microbes in a growth medium which contains the desired carbon containing feedstock as well as the other nutrients required for microbial growth, such as ammonia, salts, and trace metals. There are many variations of suitable microbial growth media, known in the art. Certain microbes, such as yeast, produce ethanol naturally and accordingly have been widely applied in the brewing industry. Other microbes, such as the bacterium, E. coli, which do not typically produce ethanol can be genetically engineered to alter their biochemical pathways so they are capable of ethanol production. In each case, ethanol produced by microbes is excreted into the extracellular culture medium, whether in liquid culture or on solid growth media. The secreted ethanol can then be detected and quantified by any suitable means. It is highly desirable to employ a detection method that can at least partially quantify the ethanol produced by each individual microbe or the microbes in an individual microbial colony. As such, a preferred screening method is one which is applied in a solid phase screen, in which a very large number of individual microbal colonies can be easily separated. This offers a much higher throughput compared to a solely liquid phase screen in which samples must be manually separated and measured in liquid assay. The present colorimetric assay can be readily applied to detect a bioalcohol such as ethanol in a liquid phase or solid phase assay. The alcohol oxidase enzyme is highly active in a liquid assay or when present in a solid assay medium.

The colorimetric assay of the present invention relies on detecting the hydrogen peroxide by-product of a successful oxidase reaction upon an alcohol. The hydrogen peroxide is reacted upon by a second enzyme, a peroxidase such as Horseradish Peroxidase, and a peroxidase co-substrate such as, but not limited to, 4-aminoantipyrine/2,4,6-trobromo-3-hydroxybenzoic acid (4-AAP/TBHBA), 2,2′-azino-bis(3-ethylbenzthiazoline-6-sulfonic acid) (ABTS), or 3,3′-diaminobenzidine tetrahydrochloride (DAB). Since the co-substrate is responsible for generating color, any compound capable of generating a colorimetric reaction when reacted with hydrogen peroxide may be used as a co-substrate in the present invention.

The colorimetric reaction of the present invention can be applied in a novel, extremely high-throughput manner to detect individual microbial isolates (such as colonies growing on a Petri plate) that are producing a desired bioalcohol such as, but not limited to, an aliphatic alcohols having 1 to 4 carbons. Consequently, the present invention contemplates screening microbes that produce bioalcohols such as, but not limited to, methanol, ethanol, butanol, 1,2 propanediol, or 1,3 propanediol. For example, microbial colonies of a microbe being screened can be subjected to random mutation and then plated on a growth medium containing the assay solution of the present invention. Any mutants that have randomly improved their ability to produce ethanol will be detected by their generation of color. Generation of more color or faster color than progenitor organisms is an indicator of a superior bioalcohol producing microbe. It is also significant that no color is generated by the reaction of the assay components with the carbon containing feedstock used for the growth of the microbe. Highly desirable carbon sources, or feedstock, for fermentative production of ethanol include, but are not limited to, glucose derived from corn or cellulosic biomass, or glycerol derived from biodiesel manufacture. For this reason the present invention verifies that the alcohol oxidase has no activity towards glucose and glycerol.

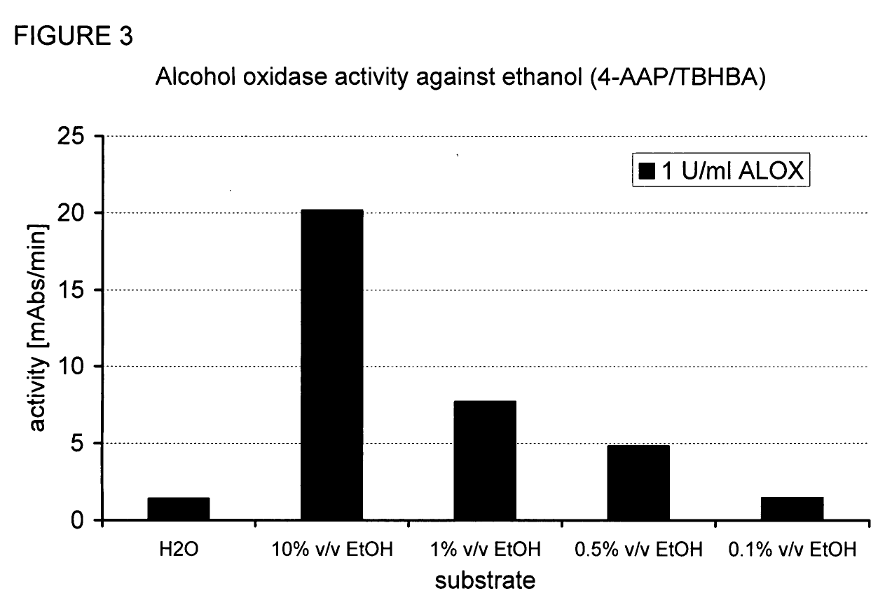

As demonstrated in Examples 1 and 2, both liquid assays were used to test the color generated when Pichia pastoris alcohol oxidase was tested for activity towards various alcohols as substrates as well as a water control. The assays were conducted using 96 well plates. A significant aspect of the present invention is that the colorimetric assay, using the Pichia pastoris alcohol oxidase, shows no detectable activity towards glucose or glycerol. As discussed above, this is important as this reaction can now be used to screen for ethanol production from a microbe which is grown on glucose or glycerol (glycerine) as the sole carbon source. The carbon source will not generate color, but the ethanol product will. FIG. 3 demonstrates that the present invention has sufficient sensitivity to detect ethanol even at a very low levels between 0.5% and 0.1% despite a low level of background activity (water control).

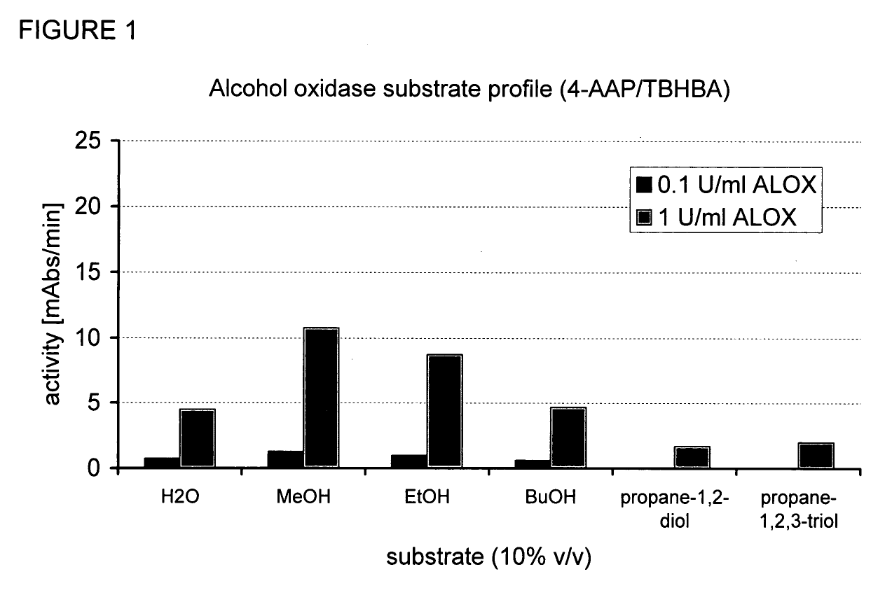

A complete description of the assays of the present invention are described in Examples 1-3 below, as well as in FIGS. 1-3. An important aspect of having three complementary assays is that each assay can detect hydrogen peroxide using different HRP co-substrates that generate different colors. This offers more versatility in colorimetric detection by a machine or by the human eye. The reaction can be performed in a spectrophotometer or in a 96 well plate as known in the art. The absorbance is measured at 510 nm, 30° C. using a VERSAmax plate reader from Molecular Devices.

### EXAMPLE 1

### Detection of Biofuel Producing Microbes Using Horseradish Peroxidase (HRP) Co-Substrate: 4-AAP/TBHBA*

### *4-AAP 4-Aminoantipyrine

### TBHBA 2,4,6-Trobromo-3-hydroxybenzoic acid

### Assay:

### 193.4 μl assay solution+6.6 μl alcohol substrate or water control

Assay solution (20X):

| | | |
|---|---|---|
| 100.0 | μl | 2x 4-AAP/TBHBA mixture* |
| 2.0 | μl | Horseradish Peroxidase from Sigma (5 mg/ml) |
| 84.8 | μl | H₂O |
| 6.6 | μl | alcohol oxidase (0.1 U/ml) |

*2x 4-AAP/TBHBA mixture (50 ml):

| | | |
|---|---|---|
| 10.00 | ml | KPhos (1M pH 7.6-8.0) |
| 1.00 | ml | TBHBA 2% in DMSO (20 mg/ml DMSO) |
| 15.24 | mg | 4-AAP (1M, M: 203.25 g/mol) |
| 39.00 | ml | H₂O |

The results of Example 1 are depicted in FIG. 1, which is a graphical representation of the substrate specificity of alcohol oxidase from P. pastoris. Alcohol oxidase was assayed against various alcohols (10% v/v), namely methanol, ethanol, butanol, 1,2-propanediol, and 1,3-propanediol, using a colorimetric screen and 4-AAP/TBHBA as the HRP co-substrate as described by the present invention. The activity data is shown in mAbs/min for two different alcohol oxidase concentrations. No activity is detected towards glycerol (glycerine) or glucose (not shown in the graph), which serve as microbial feedstocks for biofuel production.

### EXAMPLE 2

### Detection of Biofuel Producing Microbes Using Horseradish Peroxidase (HRP) Co-Substrate: ABTS*

### *ABTS: 2,2′-Azino-bis(3Ethylbenzthiazoline-6-Sulfonic Acid)

Assay solution (20X):

| | | |
|---|---|---|
| 185.0 | μl | ABTS (1 tablet/10 ml 100 mM Kphos pH 7.5) |
| 1.0 | μl | Horseradish Peroxidase from Sigma (1 mg/ml) |
| 0.8 | μl | H₂O |
| 6.6 | μl | alcohol oxidase (0.1 U/ml) |

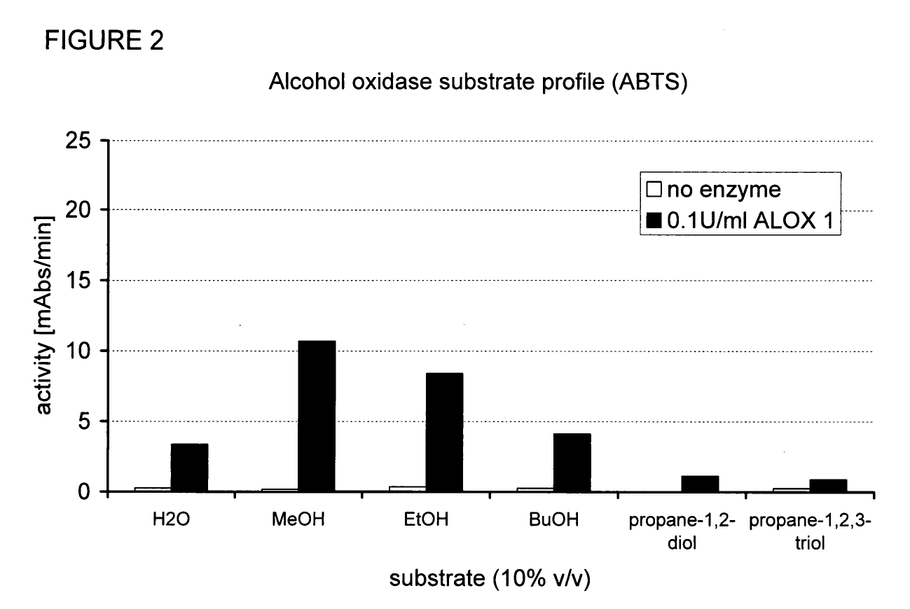

The results of Example 2 are depicted in FIG. 2, which is a graphical representation of the substrate specificity of alcohol oxidase from P. pastoris. Alcohol oxidase was assayed against various alcohols (10% v/v), namely methanol, ethanol, butanol, 1,2-propanediol, and 1,3-propanediol, using a colorimetric screen and ABTS as the HRP co-substrate described by the present invention. Activity is shown in mAbs/min using an alcohol oxidase concentration of 0.1 U/ml. No activity is detected towards glycerol (glycerine) or glucose (not shown in the graph), which serve as microbial feedstock for biofuel production.

FIG. 3 further depicts a graphical representation of the substrate specificity of alcohol oxidase from P. pastoris. Alcohol oxidase (ALOX) was assayed against ethanol at different concentrations (0-10% v/v) using a colorimetric screen and 4-AAP/TBHBA as the HRP co-substrate described by the present invention. Activity is shown in mAbs/min for an alcohol oxidase concentration of 1 U/ml.

### EXAMPLE 3

### Detection of Biofuel Producing Microbes Using Horseradish Peroxidase (HRP) Co-Substrate: DAB* in a Solid Phase Assay

### *DAB: 3,3′-Diaminobenzidine tetrahydrochloride

Assay: 5 ml assay solution+15 ml LB-agar containing 10% glucose and required antibiotic (LB-agar is cooled to 40° C. to avoid inactiviation of alcohol oxidase). The mixture is oured into a petri dish and allowed to solidify. The microbial colonies are plated on the surface of the agar and cultured at 30-37° C. in an incubator. Production of the target bioalcohol by individual colonies can then be detected by observation of color development.

Assay solution:

| | |
|---|---|
| ½ tablet | DAB in 10 ml water (sterile filtered) |
| 60.0 μl | Horseradish Peroxidase from Sigma (5 mg/ml) |
| 20.0 μl | Phosphate buffer (1M, pH 7.6) |
| 4.0 μl | alcohol oxidase (1.3 U/μl) |

The present invention further contemplates cloning a gene encoding an alcohol oxidase from a microbial source such as, but not limited to, Pichia or Saccharomyces to facilitate procedures of directed evolution. This technique can apply the encoded alcohol oxidase, using methods well known in the art, to identify variants with increased activity towards target compounds of interest, such as butanol. Such variants may be identified by introducing a gene library that encodes random variants of the Pichia alcohol oxidase into a suitable host organism, and directly screens for individual isolates that produce increased color in the presence of butanol. Such variants may then be more useful than the wild type alcohol oxidase in screening methods to identify strains producing butanol more efficiently.

Certain modifications and improvements will occur to those skilled in the art upon a reading of the foregoing description. The above-mentioned examples are provided to serve the purpose of clarifying aspects of the invention and it will be apparent to one skilled in the art that they do not serve to limit the scope of the invention. All modifications and improvements have been deleted herein for the sake of conciseness and readability but are properly within the scope of the present invention.

What is claimed is:

1. A method for detecting biofuel producing microbes, the method comprising:
a) providing a microbe;
b) providing an assay solution containing an alcohol oxidase, a peroxidase, and a peroxidase co-substrate; and
c) contacting the microbe with the assay solution to produce a colorimetric reaction to screen for the presence of a biofuel producing microbe.

2. The method of claim 1, wherein the alcohol oxidase is obtained from yeast.

3. The method of claim 2, wherein the yeast comprises Pichia, Pichia pastoris, Pichia angusta, or Saccharomyces.

4. The method of claim 1, wherein the peroxidase is Horseradish Peroxidase.

5. The method of claim 1, wherein the peroxidase co-substrate is 4-aminoantipyrine/2,4,6-trobromo-3-hydroxybenzoic acid.

6. The method of claim 1, wherein the peroxidase co-substrate is 2,2′-azino-bis(3-ethylbenzthiazoline-6-sulfonic acid).

7. The method of claim 1, wherein the peroxidase co-substrate is 3,3′-diaminobenzidine tetrahydrochloride.

8. The method of claim 1, wherein the peroxidase co-substrate is a compound capable of generating a colorimetric reaction when reacted with hydrogen peroxide.

9. The method of claim 1, wherein the microbe is cultured in a carbon containing feedstock.

10. The method of claim 9, wherein the feedstock comprises glycerol, glucose, or cellulosic biomass.

11. The method of claim 9, wherein the step of culturing the microbe further comprises producing a bioalcohol.

12. The method of claim 11, wherein the bioalcohol is an aliphatic alcohol having from 1 to 4 carbon atoms.

13. The method of claim 11, wherein the bioalcohol comprises methanol, ethanol, butanol, 1,2 propanediol, or 1,3 propanediol.

14. The method of claim 1, wherein screening for the biofuel producing microbe is conducted in a solid phase assay.

15. The method of claim 1, wherein screening for the biofuel producing microbe is conducted in a liquid phase assay.

16. A method for detecting biofuel producing microbes, the method comprising:
a) providing a microbe;
b) providing a carbon containing feedstock;
c) providing an assay solution containing an alcohol oxidase, a peroxidase, and a peroxidase co-substrate;
d) culturing the microbe in the feedstock as part of a culture medium; and
e) contacting the culture medium with the assay solution to produce a colorimetric reaction to screen for a biofuel producing microbe.

17. The method of claim 16, wherein the step of culturing the microbe produces a bioalcohol.

18. The method of claim 16, wherein the feedstock does not produce a colorimetric reaction.

19. A method for detecting biofuel producing microbes, the method comprising:
a) providing a microbe;
b) providing an assay solution containing an alcohol oxidase, a peroxidase, and a peroxidase co-substrate;
c) contacting the microbe with the assay solution to produce a detectable amount of hydrogen peroxide; and
d) detecting the amount of hydrogen peroxide produced using a colorimetric reaction to screen for a biofuel producing microbe.

20. A kit for detecting the presence of a biofuel producing microbe, the kit comprising:
a container containing an assay solution, the assay containing an alcohol oxidase, a peroxidase, and a peroxidase co-substrate;
a set of instructions on how to use the kit to detect a biofuel producing microbe; and
a packaging holding the container and the instructions.
